(19) United States
(12) Patent Application Publication
(10) Pub. No.: US 2025/0298113 A1
(43) Pub. Date: Sep. 25, 2025

(54) DEMAND-ORIENTED FUNCTIONAL TESTS FOR THE EFFICIENT DETECTION OF SPORADIC ERRORS BASED ON A PATIENT-SPECIFIC RISK ASSESSMENT

(71) Applicant: Siemens Healthineers AG, Forchheim (DE)

(72) Inventor: Thomas Beck, Dormitz (DE)

(21) Appl. No.: 19/083,417

(22) Filed: Mar. 18, 2025

(30) Foreign Application Priority Data
Mar. 19, 2024 (DE) 10 2024 202 563.1

(51) Int. Cl. G01R 33/58; A61B 5/055; G16H 10/60; G16H 30/40

(52) U.S. Cl. CPC G01R 33/58; A61B 5/055; G16H 10/60; G16H 30/40

(57) ABSTRACT

Methods for performing a magnetic resonance examination on a patient with a magnetic resonance apparatus, a magnetic resonance apparatus, and a computer program product are provided. According to the method, patient-specific information about the patient is provided. The patient-specific information about the patient ascertains whether at least one latent error test is to be performed on the magnetic resonance apparatus. If it is ascertained that the at least one latent error test to check the magnetic resonance apparatus is to be performed, the at least one latent error test is performed. The magnetic resonance examination is performed depending on a result of the at least one latent error test.

This application claims the benefit of German Patent Application No. DE 10 2024 202 563.1, filed on Mar. 19, 2024, which is hereby incorporated by reference in its entirety.

### BACKGROUND

The present embodiments relate to a method for performing a magnetic resonance examination on a patient with a magnetic resonance apparatus, a magnetic resonance apparatus, and a computer program product.

During a magnetic resonance examination on a patient (e.g., during the performance of magnetic resonance imaging (MRI)), magnetic fields (e.g., gradient fields) and radio-frequency signals or radio-frequency fields are usually used to capture magnetic resonance signals by a magnetic resonance apparatus according to a scan protocol. The magnetic resonance apparatus may have a gradient coil unit to generate the gradient fields. Further, the magnetic resonance apparatus may include a radio-frequency antenna unit with which the radio-frequency signals may be generated in order to excite atomic nuclei.

As the performance of the magnetic resonance apparatus increases with regard to gradient strength and radio-frequency power, risks for the patient may arise (e.g., through stimulation of the patient's heart muscle as a result of induced voltage in the tissue due to strong temporal magnetic field gradients and/or overheating of the patient's tissue due to strong radiated radio-frequency power).

To make matters worse, any implants in the patient's body may bundle both magnetic fields and radio-frequency fields locally (e.g., due to their effect as a passive antenna). This may lead to a local field increase so that higher field strengths may occur in the patient's body than in cases without implants.

In the event of a serious risk (e.g., “serious injury or death of a single person”), suitable mitigation measures are to be implemented (e.g., if the probability of occurrence is unacceptably high). The aim of these measures is to reduce the probability of occurrence and/or the severity of the risk.

One possibility for implementing a mitigation measure is to check a control path that controls the magnetic resonance examination on the patient in a separate test path, which may be referred to as a protect path. The task of the control path is to specify or influence a setpoint value of a control variable (e.g., for controlling the gradient coil unit and/or radio-frequency antenna unit). Publication DE102020206063A1 discloses by way of example a magnetic resonance apparatus with a control path and two protect paths.

In the test path, an actual value measured at the respective point in time is captured (e.g., completely independently thereof) and compared with the setpoint value. If there is a deviation between the target value and the actual value, a corresponding error response is triggered. The safe state may be assumed, which generally provides a controlled scan abort. However, it is possible that a failure of the test path is not immediately recognizable. Therefore, undetected latent errors may occur (e.g., if the test path no longer fulfils its test function correctly due to a sensor error).

To detect these latent errors, latent error tests are performed on the magnetic resonance apparatus at regular intervals in which the functionality of the test path is explicitly triggered. For this purpose, for example, target values that exceed permissible limit values may be deliberately set, thereby triggering the test path. The time required to perform such a latent error test may well be in the range of minutes. From the user's point of view, it is therefore highly desirable to plan the performance of the tests in such a way that the tests have no impact, or as little impact as possible, on patients.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, the correct (e.g., safe) operation of a magnetic resonance apparatus or the performance of a magnetic resonance examination is improved.

A computer-implemented method for performing a magnetic resonance examination on a patient with a magnetic resonance apparatus is provided. Herein, patient-specific information about the patient is provided. The patient-specific information about the patient is used to ascertain whether at least one latent error test should be performed on the magnetic resonance apparatus. If it is ascertained that the at least one latent error test to check the magnetic resonance apparatus should be performed, the at least one latent error test is performed. The magnetic resonance examination is performed in dependence on a result of the at least one latent error test.

For example, the patient-specific information about the patient is provided by a providing unit. For example, ascertaining whether at least one latent error test on the magnetic resonance apparatus should be performed is provided by an ascertaining unit. The providing unit and/or the ascertaining unit may, for example, include one or more processors and/or one or more memory modules. The providing unit and/or the ascertaining unit may, for example, be part of a system control unit of the magnetic resonance apparatus.

In one embodiment, ascertaining whether at least one latent error test on the magnetic resonance apparatus should be performed includes ascertaining a patient-specific risk that may be mitigated by the at least one latent error test based on the patient-specific information. For example, it is ascertained whether there is a patient-specific risk that requires functionally safe monitoring. In one embodiment, the patient-specific risk of the magnetic resonance examination is ascertained in dependence on a probability of error occurrence (e.g., low, medium, or high probability) and/or the severity of error consequences (e.g., minor injury, serious injury or even death of the patient). The higher the probability of error occurrence and/or the severity of error consequences, the more likely it is that at least one latent error test should be performed. If, for example, multiplying a measure of the probability of error occurrence by a measure of the severity of error consequences produces a value that is above a prespecified threshold value, a latent error test should be performed to check the magnetic resonance apparatus.

In one embodiment, the regular operation of the magnetic resonance apparatus is only interrupted to the smallest possible extent by the patient-specific or demand-oriented execution of latent error tests. In contrast to the prior art, latent error tests are performed at regular intervals independently of the patient-specific risk. Therefore, the method of the present embodiments may reduce the overall time required for the execution of the latent error tests. The method is, for example, advantageous if the risk that is mitigated with the at least one latent error test is only relevant for a relatively small proportion of patients and this patient group is not evenly distributed across the patient population.

In one embodiment, the magnetic resonance examination is only performed if the result of the at least one latent error test indicates a correct (e.g., safe) operation of the magnetic resonance examination and/or a correct (e.g., safe) performance of the magnetic resonance examination.

In one embodiment, the at least one latent error test ascertains whether at least one test path of the magnetic resonance apparatus is functioning correctly. For example, a positive result of the at least one latent error test provides that any sensors in a test path of the magnetic resonance apparatus are functioning correctly. If, for example, the result of the latent error test was to be that a sensor in the test path has failed, the patient may not undergo a magnetic resonance examination.

The at least one test path may be configured to test at least one control path of the magnetic resonance apparatus during the magnetic resonance examination (e.g., a magnetic resonance scan). The at least one test path, for example, includes elements that contribute to monitoring the safety-relevant function. For example, the at least one test path includes sensors and/or detectors for capturing at least one safety-relevant parameter of the magnetic resonance apparatus. The at least one safety-relevant parameter may, for example, include safety-relevant system variables of the magnetic resonance apparatus (e.g., a gradient current).

The at least one control path of the magnetic resonance apparatus may be configured to specify or influence a setpoint value of a control variable (e.g., for controlling the gradient coil unit and/or radio-frequency antenna unit). In the test path, an actual value measured at the respective time is captured (e.g., completely independently thereof) and compared with the setpoint value. If there is a deviation between the target value and the actual value, a corresponding error response is triggered. The safe state is assumed, which generally provides a controlled abortion of the examination (e.g., abortion of the scan).

In one embodiment, the patient-specific information includes information about an implant in the patient. For example, the information about the implant in the patient includes at least one limit value to be complied with. The at least one limit value to be complied with may, for example, be a maximum gradient slew rate. The severity of the error consequences in patients with implants may, for example, be significantly higher than in patients without implants.

For example, some patients have implants that are classed as “MR conditional.” “MR conditional” implants are products with proven safety in magnetic resonance examinations within defined conditions (e.g., if specific limit values are complied with). These limit values are often significantly lower than for patients without implants. This is, for example, the case for patients with pacemakers, for whom the limit value for the maximum gradient slew rate is 125 T/m/s instead of 200 T/m/s. Another example is cochlear implants, which in some cases also have limit values well below 200 T/m/s.

The more sporadic and critical the risks for individual patients are, the greater the time gain that may be achieved by the method of the present embodiments. This is, for example, the case with relatively rare “MR conditional” implants with maximum gradient slew rates well below 200 T/m/s.

In one embodiment, during the performance of the at least one latent error test, at least one malfunction of the magnetic resonance apparatus to be checked is triggered, and it is checked whether the at least one malfunction of the magnetic resonance apparatus to be checked is detected.

In latent error tests, for example, the functionality of the test path is explicitly triggered. For this purpose, it is, for example, possible for target values that exceed permissible limit values to be deliberately set, thereby triggering the test path.

In one embodiment, the at least one malfunction to be checked relates to the safety of the patient and/or correct functioning of the magnetic resonance apparatus during the magnetic resonance examination.

In addition to functionally safe monitoring of a control task, patient-specific latent error tests may also be applied to control tasks that are not implemented with functional safety. Similarly, the aim is then also to reduce the probability of error occurrence by ensuring that the magnetic resonance apparatus is functioning correctly before scanning a patient (e.g., in the case of patient-specific increased risks).

In one embodiment, the magnetic resonance examination is performed immediately after the at least one latent error test on the magnetic resonance apparatus has been performed. In one embodiment, in the case of particularly critical magnetic resonance scans, the correct function of the magnetic resonance apparatus is ensured directly before the examination. Taking into account the mean time to failure (MTTF), the probability of failure of monitoring hardware or a test path may be considerably lower if the correct function is ensured directly before the examination rather than at fixed time intervals.

In one embodiment, if it is ascertained that at least one latent error test on the magnetic resonance apparatus should be performed, the method includes a determination of the at least one malfunction to be checked and/or a parameterization of the at least one latent error test on the magnetic resonance apparatus. In one embodiment, the at least one latent error test is configured such that the at least one latent error test optimally minimizes the patient-specific risk to be mitigated.

Further, a magnetic resonance apparatus is provided that is configured to execute a method as described above for performing a magnetic resonance examination on a patient with the magnetic resonance apparatus. The advantages of the magnetic resonance apparatus substantially correspond to the advantages of the method for performing a magnetic resonance examination on a patient with the magnetic resonance apparatus as described in detail above. Features, advantages, or alternative embodiments described herein may also be transferred to the other subject matter, and vice versa.

Further, a computer program product is provided that includes a program and may be loaded directly into a memory of a programmable system control unit of a magnetic resonance apparatus and has program means (e.g., libraries and auxiliary functions) for executing a method of the present embodiments when the computer program product is executed in the system control unit of the magnetic resonance apparatus. Herein, the computer program product may include software with a source code that still has to be compiled and linked or only has to be interpreted, or an executable software code that only has to be loaded into the system control unit for execution.

The computer program product enables the method of the present embodiments to be executed quickly, identically repeatedly, and robustly. The computer program product may be configured such that the computer program product may execute the proposed method acts using the system control unit. Herein, the system control unit in each case fulfills the requisite conditions, such as, for example, an appropriate random-access memory, an appropriate graphics card, or an appropriate logic unit, so that the respective method steps may be executed efficiently.

The computer program product is, for example, stored on a computer readable medium or held on a network or server from where the computer program product may be loaded into the processor of a local system control unit that may be directly connected to the magnetic resonance apparatus or be embodied as part of the magnetic resonance apparatus. Further, control information of the computer program product may be stored on an electronically readable data carrier. The control information of the electronically readable data carrier may be embodied to perform a method of the present embodiments when the data carrier is used in a system control unit of a magnetic resonance apparatus.

Examples of electronically readable data carriers are DVDs, magnetic tapes, or USB sticks on which electronically readable control information (e.g., software) is stored. When this control information is read from the data carrier and stored in a system control unit of the magnetic resonance apparatus, all embodiments of the above-described methods may be performed.

Further advantages, features, and details of the invention emerge from the example embodiments described below and with reference to the drawings. Corresponding parts are given the same reference symbols in all figures.

### BRIEF DESCRIPTION OF THE DRAWINGS

FIG. 1 shows a magnetic resonance apparatus in a schematic representation; and

FIG. 2 shows a flowchart of a method for performing a magnetic resonance examination on a patient with the magnetic resonance apparatus.

### DETAILED DESCRIPTION

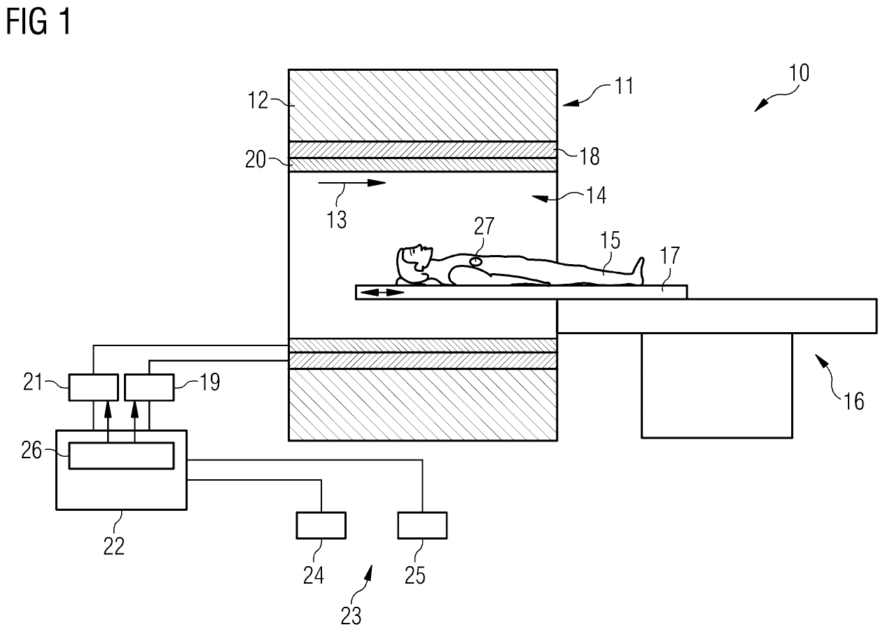

FIG. 1 shows a schematic representation of a magnetic resonance apparatus 10. The magnetic resonance apparatus 10 includes a magnet unit 11 that has a main magnet 12 for generating a strong and, for example, temporally constant main magnetic field 13. In addition, the magnetic resonance apparatus 10 includes a patient receiving region 14 for receiving a patient 15. In the present example embodiment, the patient receiving region 14 is configured as cylindrical and is surrounded in a cylindrical shape by the magnet unit 11 in a circumferential direction. In principle, however, an embodiment of the patient receiving region 14 deviating therefrom may be provided. The patient 15 may be pushed into the patient receiving region 14 by a patient support apparatus 16 of the magnetic resonance apparatus 10. For this purpose, the patient support apparatus 16 has a patient table 17 configured to be movable within the patient receiving region 14.

The magnet unit 11 also has a gradient coil unit 18 for generating magnetic field gradients that are used for spatial encoding during imaging. The gradient coil unit 18 is controlled by a gradient control unit 19 of the magnetic resonance apparatus 10. The magnet unit 11 further includes a radio-frequency antenna unit 20 that, in the present example embodiment, is configured as a body coil permanently integrated into the magnetic resonance apparatus 10. The radio-frequency antenna unit 20 is controlled by a radio-frequency antenna control unit 21 of the magnetic resonance apparatus 10 and radiates radio-frequency magnetic resonance sequences into an examination space that is substantially formed by a patient receiving region 14 of the magnetic resonance apparatus 10. As a result, the main magnetic field 13 generated by the main magnet 12 excites atomic nuclei. Magnetic resonance signals are generated by relaxation of the excited atomic nuclei. The radio-frequency antenna unit 20 is configured to receive the magnetic resonance signals.

The magnetic resonance apparatus 10 has a system control unit 22 for controlling the main magnet 12, the gradient control unit 19, and for controlling the radio-frequency antenna control unit 21. The system control unit 22 centrally controls the magnetic resonance apparatus 10, such as, for example, performing a predetermined imaging gradient echo sequence. In addition, the system control unit 22 includes an evaluation unit (not shown in further detail) for evaluating the magnetic resonance signals captured during the magnetic resonance examination. Further, the magnetic resonance apparatus 10 includes a user interface 23 that is connected to the system control unit 22. Control information, such as, for example, imaging parameters and reconstructed magnetic resonance images, may be displayed on a display unit 24 (e.g., on at least one monitor) of the user interface 23 for medical operators. Further, the user interface 23 has an input unit 25 by which information and/or parameters may be entered by medical operators during a scanning procedure.

The system control unit 22 includes a test unit 26 that represents a test path of the magnetic resonance apparatus 10. The test path provides independent monitoring of the control of the magnetic resonance apparatus (e.g., the gradient coil unit 18 and gradient control unit 19 and the radio-frequency antenna unit 20 and radio-frequency antenna control unit 21, which represent a control path (or a part thereof)). In one embodiment, the test unit 26 recognizes whether any limit values are exceeded by the gradient coil unit 18 and gradient control unit 19 or the radio-frequency antenna unit 20 and radio-frequency antenna control unit 21. These limit values may, for example, relate to the safety of the patient 15, such as, for example, a specific absorption rate (SAR) exposure and/or a peripheral nerve stimulation (PNS) exposure. In this case, the patient 15 has an implant 27. This may result in higher requirements for patient safety (e.g., the maximal permissible values may be lower).

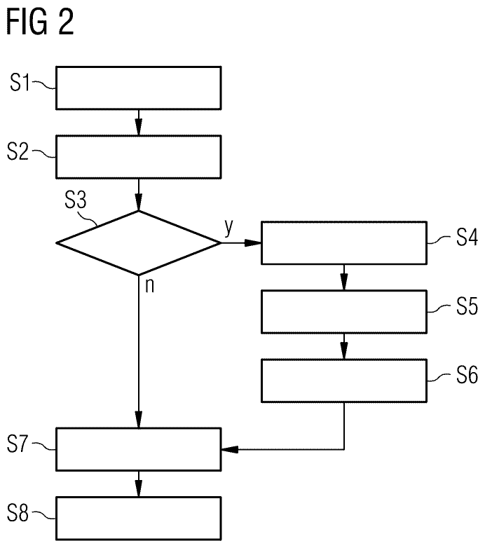

FIG. 2 shows a possible sequence of a method for performing a magnetic resonance examination on the patient 15 with the magnetic resonance apparatus 10.

In S1, the patient 15 is registered on the magnetic resonance apparatus 10. Herein, information about the patient 15 is captured, for example. The information about the patient 15 may, for example, be captured by an operator of the magnetic resonance apparatus 10 with the aid of the user interface 23. The information about the patient 15 may, for example, include the height, weight, age, gender, and/or other features of the patient 15. Such other features may, for example, include the fact as to whether or not the patient 15 is wearing an implant 27. For example, the type of implant (e.g., “MR conditional”) and/or limit values to be complied with (e.g., for B1+rms, SAR, gradient slew rate, static field gradient) may be captured.

Part of or all of this information captured about the patient 15 may be provided as patient-specific information in S2. In S3, the patient-specific information provided in S2 is used to ascertain whether or not at least one latent error test should be performed to check the magnetic resonance apparatus (e.g., y=yes, n=no).

For example, in S3, a check is performed as to whether there are any patient-specific risks that require functionally safe monitoring and/or checking. If this is the case, the demand-oriented latent error tests to be carried out are determined in S4. These latent error tests are then parameterized in S5 and performed in S6.

In the latent error test, for example, a possible malfunction and/or an exceeded limit value of the magnetic resonance apparatus 10 is intentionally triggered, and it is then checked whether the malfunction or the exceeded limit value is also detected. The malfunction to be checked may, for example, relate to the safety of the patient 15 and/or a correct function of the magnetic resonance apparatus 10 during the magnetic resonance examination.

Depending on the result of these latent error tests, the magnetic resonance examination is continued or not continued in S7. If the magnetic resonance examination is continued, further preparations for the magnetic resonance scan may be carried out in S7, for example, before the actual magnetic resonance examination (e.g., the magnetic resonance scan) is performed on the patient 15 in S8.

In one embodiment, provided that the result of the latent error test indicates that the magnetic resonance device 10 is operating safely, the magnetic resonance examination in S8 is performed immediately after the result according to S6 has been ascertained and, if necessary, further preparations for the magnetic resonance scan have been made in S7. The probability of failure of the monitoring hardware under consideration is significantly lower if the correct function is ensured directly before the examination and not at fixed time intervals, such as, for example, a certain number of days.

The method of the present embodiments results in a key advantage for system functions that may result in a very different risk assessment for different patient groups.

A specific variant of the method depicted in S2 is described in more detail below. This entails a magnetic resonance examination on a patient 15 with an “MR conditional” implant. In such a scan, the gradient slew rate is limited.

The vast majority of “MR conditional” implants for which it is necessary to limit the gradient slew rate have a limit value to be monitored of 200 T/m/s. Today's standard magnetic resonance apparatuses often have an achievable maximum gradient slew rate of 200 T/m/s.

In such a case, if additional factors such as, for example, manufacturing tolerances and fluctuations in the power supply are taken into account, there is a comparatively low probability of the patient suffering serious harm during a magnetic resonance examination. It may therefore be the case that it is not necessary to perform latent error tests for this type of magnetic resonance examination.

However, the situation is different if a patient has an “MR conditional” implant with a significantly lower limit value (e.g., 100 T/m/s). In this case, if there is a system malfunction, it is not only possible that the permissible implant limit value may be exceeded due to manufacturing tolerances and fluctuations in the power supply, but the magnetic resonance apparatus 10 may also be configured to exceed the permissible limit value of 100 T/m/s in the case of an initial error by 100 percent. Thus, the patient-specific properties may result in a different risk assessment. The method described here may provide that in this case, the correct functionality of the limit value monitoring is provided before the demand-oriented scan is carried out.

Reference is made once again to the fact that the methods described in detail above and the magnetic resonance apparatus illustrated are merely examples of embodiments that may be modified by the person skilled in the art in a wide variety of ways without departing from the scope of the invention. Further, the use of the indefinite articles “a” or “an” does not preclude the possibility that the features in question may also be present on a multiple basis. Similarly, the term “unit” does not preclude the possibility that the components in question may consist of a plurality of interacting subcomponents that may also be spatially distributed. Independent of the grammatical term usage, individuals with male, female, or other gender identities are included within the term.

The elements and features recited in the appended claims may be combined in different ways to produce new claims that likewise fall within the scope of the present embodiments. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A method for performing a magnetic resonance examination on a patient with a magnetic resonance apparatus, the method being computer-implemented and comprising:
providing patient-specific information about the patient;
ascertaining whether at least one latent error test is to be performed to check the magnetic resonance apparatus using the patient-specific information;
when it is ascertained that the at least one latent error test is to be performed:
performing the at least one latent error test on the magnetic resonance apparatus; and
performing the magnetic resonance examination in dependence on a result of the at least one latent error test.

2. The method of claim 1, wherein the patient-specific information comprises information about an implant in the patient.

3. The method of claim 2, wherein the information about the implant in the patient comprises at least one limit value to be complied with.

4. The method of claim 3, wherein the at least one limit value to be complied with includes a maximum gradient slew rate.

5. The method of claim 1, wherein ascertaining whether at least one latent error test should be performed comprises ascertaining a patient-specific risk of the magnetic resonance examination that is mitigatable by the at least one latent error test based on the patient-specific information.

6. The method of claim 5, wherein the patient-specific risk of the magnetic resonance examination is ascertained depending on a probability of error occurrence, severity of error consequences, or a combination thereof.

7. The method of claim 1, wherein the at least one latent error test comprises:
triggering at least one malfunction of the magnetic resonance apparatus to be checked; and
checking whether the at least one malfunction of the magnetic resonance apparatus to be checked is detected, a corresponding response is triggered, or a combination thereof.

8. The method of claim 7, wherein the at least one malfunction to be checked relates to safety of the patient, correct function of the magnetic resonance apparatus during the magnetic resonance examination, or a combination thereof.

9. The method of claim 1, wherein the magnetic resonance examination is performed immediately after the at least one latent error test has been performed.

10. A magnetic resonance apparatus configured to perform a magnetic resonance examination on a patient, the magnetic resonance apparatus comprising:
a processor configured to:
provide patient-specific information about the patient;
ascertain whether at least one latent error test is to be performed to check the magnetic resonance apparatus using the patient-specific information;
when it is ascertained that the at least one latent error test is to be performed:
perform the at least one latent error test on the magnetic resonance apparatus; and
perform the magnetic resonance examination in dependence on a result of the at least one latent error test.

11. In a non-transitory computer-readable storage medium that stores instructions executable by one or more processors to perform a magnetic resonance examination on a patient with a magnetic resonance apparatus, the instructions comprising:
providing patient-specific information about the patient;
ascertaining whether at least one latent error test is to be performed to check the magnetic resonance apparatus using the patient-specific information;
when it is ascertained that the at least one latent error test is to be performed:
performing the at least one latent error test on the magnetic resonance apparatus; and
performing the magnetic resonance examination in dependence on a result of the at least one latent error test.

12. The non-transitory computer-readable storage medium of claim 11, wherein the patient-specific information comprises information about an implant in the patient.

13. The non-transitory computer-readable storage medium of claim 12, wherein the information about the implant in the patient comprises at least one limit value to be complied with.

14. The non-transitory computer-readable storage medium of claim 13, wherein the at least one limit value to be complied with includes a maximum gradient slew rate.

15. The non-transitory computer-readable storage medium of claim 11, wherein ascertaining whether at least one latent error test should be performed comprises ascertaining a patient-specific risk of the magnetic resonance examination that is mitigatable by the at least one latent error test based on the patient-specific information.

16. The non-transitory computer-readable storage medium of claim 15, wherein the patient-specific risk of the magnetic resonance examination is ascertained depending on a probability of error occurrence, severity of error consequences, or a combination thereof.

17. The non-transitory computer-readable storage medium of claim 11, wherein the at least one latent error test comprises:
triggering at least one malfunction of the magnetic resonance apparatus to be checked; and
checking whether the at least one malfunction of the magnetic resonance apparatus to be checked is detected, a corresponding response is triggered, or a combination thereof.

18. The non-transitory computer-readable storage medium of claim 17, wherein the at least one malfunction to be checked relates to safety of the patient, correct function of the magnetic resonance apparatus during the magnetic resonance examination, or a combination thereof.

19. The non-transitory computer-readable storage medium of claim 11, wherein the magnetic resonance examination is performed immediately after the at least one latent error test has been performed.
